(19) United States
(12) Patent Application Publication
(10) Pub. No.: US 2006/0177426 A1
(43) Pub. Date: Aug. 10, 2006

(54) Method of preserving lyophilized microorganisms for transport, storage and recovery of viable microorganisms

(76) Inventors: Cecil Gibson, Lexington, KY (US); Gerald Chrisope, Lake Charles, LA (US)

Correspondence Address: Cecil Gibson, 1040 Manchester St, Lexington, KY, 40508, US

(21) Appl. No.: 11/347,334

(22) Filed: Feb. 3, 2006

Related U.S. Application Data
(60) Provisional application No. 60/593,737, filed on Feb. 9, 2005.

(51) Int. Cl. A61K 35/12; A01N 1/02

(52) U.S. Cl. 424/93.7; 435/284.1

(57) ABSTRACT

Incorporation of a microbial cell suspension throughout a fibrous network provides a physical environment that allows greater removal of water during lyophilization or desiccation thereby yielding a device with improved stability and recovery of viable microbial cells. Strands of appropriate fibers in a tightly knit network absorb aqueous cell suspensions by a capillary effect rather than absorption. When vapor pressure is decreased by vacuum during lyophilization or by air moved during desiccation, the surface tension is affected at the fiber/water interface, which results in increased water removal, by a “reverse capillary” effect. Thus bound and free water removal is increased. Therefore, incorporation of the use of a network of fibers in conjunction with a preservation matrix containing sensitive microbial cells provides a means of producing a preserved product with increased stability at both extreme and routine storage temperatures and greater efficacy for the end user. The invention also provides a means for recovery of viable microbial cells by direct inoculation to solid or liquid culture media as recommended for use in performance or quality control testing of culture media, stains, identification kits, maintenance of stock cultures and in the evaluation of bacteriological procedures. Additionally, the device can be used to mimic clinical specimens in clinical or industrial proficiency testing surveys that test the ability of laboratory technologists to properly perform diagnostic procedures.

### CROSS-REFERENCES TO RELATED APPLICATIONS

This application is entitled to the benefit of Provision Patent Application Ser. No. 60/593737, filed Feb. 9, 2005.

### FIELD OF THE INVENTION

The present invention is a novel device comprised of preserved microbial cells or cellular components, specifically to an improved means of storing, transporting and recovering viable microbial cells. More particularly, the invention allows for enhanced removal of water during lyophilization or desiccation processes increasing stability of microbial cells. The invention also provides a means for recovery of viable microbial cells by direct inoculation to solid or liquid culture media as recommended for use in performance or quality control testing of culture media, stains, identification kits, maintenance of stock cultures and in the evaluation of bacteriological procedures. Additionally, the device can be used to mimic clinical specimens in clinical or industrial proficiency testing surveys that test the ability of laboratory technologists to properly perform diagnostic procedures.

### BACKGROUND TO THE INVENTION

Commercial provision of microorganisms on a global basis requires that the preserved microbial cells in a device maintain viability throughout the rigors imposed by distribution and shipping to the final destination in addition to subsequent storage at the final destination. Freeze-drying or lyophilization is generally recognized as an effective means for the preservation of microbial cells. Processes of lyophilization or freeze-drying methods for a variety of microorganisms have been described in American Type Culture Collection Methods, I. Laboratory Manual on Preservation: Freezing and Freeze-Drying, Hatt, H. (ed.), ATCC (1980).

During lyophilization, it is a standard procedure to incorporate cryoprotectants in addition to a nutritional suspending agent to minimize damage to microbial cells and maintain survival of microbial cells. Cryoprotectants used in prior art have included dried skim milk, glucose, sucrose, lactose, monosodium glutamate and bovine serum albumin. However, even addition of cryoprotectants does not always provide a solution for long-term stability and recovery of microbial cells.

Perhaps, more importantly is the removal of water during lyophilization or desiccation. If sufficient bound or unbound water is not removed during the preservation process, stability is severely compromised resulting in loss of viability of the preserved microbial cells. Insufficient removal of bound and unbound water results in residual water which enables metabolic processes to continue in the preserved cells resulting in accumulation of metabolic end-products such as acids. These accumulated metabolites lead to cell death and decreased shelf life particularly concentrated as they are in a microenvironment.

Various devices have been proposed in which microbial cell suspensions are freeze-dried or desiccated, but each has disadvantages and drawbacks in delivery of the microorganism to culture medium. These devices are packaged and stored in a variety of vessels requiring reconstitution with various liquids prior to utilization in testing procedures. See for example, U.S. Pat Nos. 5,279,964; 5,155,039; 4,672,037; 5,710,041; 6,057,151; 6,322,994

In one prior art, a plastic sleeve houses a swab that is positioned between a glass ampoule containing rehydration fluid and a pellet of lyophilized bacteria. Upon fracture of the glass ampoule, the rehydration fluid is released to reconstitute the pellet. Deficiencies for this type of system are due to the inclusion of the glass ampoule and inherit dangers of exposing users to live microorganisms during fracturing of the glass ampoule.

Another prior art relies upon the use of a loop. In this instance, users must provide their own rehydrating liquid, dip the loop into the liquid, and then apply the loop to the growth medium.

Another prior art utilizes a system of first and second vial and cap combinations, the first carrying a pre-measured quantity of rehydration fluid. The microbial suspension is dried to fixative sites on the underside of the first cap. The first cap must then be transferred to the second vial containing the rehydration fluid. Once rehydration, the organisms must then be transferred to the appropriate culture media.

It is an object of the present invention to provide an improved method of freeze-drying or desiccating microorganisms by impregnating the microbial suspension throughout a fibrous network to facilitate removal of bound and unbound water to improve survival and recovery of viable microbial cells.

It is also an object of the present invention to provide a device having a specific composition of freeze-dried microorganisms such that pre-rehydration steps are eliminated providing for direct inoculation to solid or liquid culture media.

### SUMMARY OF THE INVENTION

Incorporation of a microbial cell suspension throughout a fibrous network provides a physical environment that allows greater removal of water during lyophilization or desiccation thereby yielding a device with improved stability and recovery of viable microbial cells. Strands of appropriate fibers in a tightly knit network absorb aqueous cell suspensions by a capillary effect rather than absorption. That is, each strand is somewhat hydrophobic, certainly not truly hydrophilic. Hence, the water retentive property of the mass of fibers in the network is created by the capillary action of the small channels between the fibers in close proximity to each other. Lack of true hydrophilicity of the strands creates a degree of surface tension at the surface of each strand as the aqueous cell suspension is introduced. When vapor pressure is decreased by vacuum during lyophilization or by air moved during desiccation, the surface tension is affected at the fiber/water interface, which results in increased water removal, by a “reverse capillary” effect. Thus water removal is increased, both bound and unbound water. Residual bound water is widely known to decrease stability and shelf life of preserved microbial cells. Therefore, incorporation of the use of a network of fibers in conjunction with a preservation matrix containing sensitive microbial cells provides a means of producing a preserved product with increased stability and greater efficacy for the end user.

The present invention permits the preservation and long-term storage of preserved microbial cells at both extreme temperatures (35-37 C and 25 C) as well as refrigeration temperatures (2-8 C).

### DETAILED DESCRIPTION OF THE INVENTION

The preservation process is accomplished by impregnating an aqueous cell suspension throughout a matrix of a network of closely-knit fibers. The Fibers may include but are not limited to Dacron, nylon, rayon, cotton or other natural or synthetic fibers. Further it is assumed that the fibrous network may be attached to a shaft or some other delivery component for utility purposes. An appropriate amount of entrapped water is then removed from the cell-laden fibrous network by lyophilization or desiccation. The resulting dry fibrous network contains preserved viable microbial cells that may be efficiently rehydrated at room temperature by direct contact with selected solid or liquid culture media.

This invention is applicable to a wide variety of microorganisms, including fungi, yeasts and bacteria. Microbial cells are propagated in an appropriate culture medium. Appropriate culture medium generally contains carbon and nitrogen sources in addition to growth factors. Appropriate media are available from commercial sources. The microorganisms are incubated under optimum conditions (e.g. atmospheres and temperatures). The microbial cell growth is then harvested in the logarithmic phase of growth.

Following harvest of said microbial cells, the viable microbial cells are concentrated in a preservation medium. The preservation medium is composed of a variety of cryoprotectant agents, designed to minimize cellular damage and increase survivability of microorganisms during lyophilization and desiccation. In the broad practice of this invention, any of a wide variety of cryoprotectants can suitably be employed. Microorganisms are suspended in a preservation medium that provides protection of the cell walls during freeze-drying or desiccation and subsequent extended storage. The preservation medium contains an agent to neutralize any toxic substances that may be formed during the preservation process.

The microbial cell suspension is quantitatively added to a mass of fibers such as Dacron, nylon, rayon, cotton or some other fibrous material. This is accomplished by dispensing standardized aliquots of the said microbial suspension into sterile trays. The fibrous network of material is utilized to suspend the cell suspension throughout the network. The inoculated fibrous material undergoes the process of lyophilization or desiccation to remove water and preserve the microbial suspension.

After lyophilization or desiccation, the fibrous network is properly packaged with desiccants in a water-barrier pouch to prevent any adverse accumulation of moisture. In accordance with the method of this invention, a growing culture of the lyophilized microorganism can now be cultured upon direct contact of the fiber with a suitable liquid or solid culture medium. The method of release of the preserved microbial cells to the liquid or solid culture medium can be accomplished under room temperature conditions and without requirements of additional rehydration fluids.

The following examples are included for illustrative purposes only and are not intended to limit the scope of this invention.

### EXAMPLE 1

When using the present invention, preservation of microbial cell suspensions maintained viability without significant loss under extreme temperatures (35-37 C) for up to 28 days.

Microorganisms were recovered by direct inoculation of the fibrous network to culture media plates. No rehydration fluid was necessary for recovery of viable cells. (Table 1)

### EXAMPLE 2

When using the present invention, preservation of microbial cell suspensions maintained viability without significant loss under room temperature (30 C) for up to 6 months. Microorganisms were recovered by direct inoculation of the fibrous network to culture media plates. No rehydration fluid was necessary for recovery of viable cells. (Table 2)

### EXAMPLE 3

When using the present invention, preservation of microbial cell suspensions maintained viability without significant loss under normal storage conditions (2-8 C) for up to 15 months. Microorganisms were recovered by direct inoculation of the fibrous network to culture media plates. No rehydration fluid was necessary for recovery of viable cells. (Table 3)

### EXAMPLE 4

A comparison study was conducted to evaluate the fibrous network versus typical pellet structures for preservation of microbial cells. The same matrix, desiccation methodology and cell suspension were utilized. Results indicated that the fibrous network laden with microbial cell suspension provided greater recovery of viable cells when compared to desiccated pellets utilizing rehydration fluid for recovery. (Table 4)

TABLE 1
ACCELERATED STUDIES (Storage Temperature: 35-37 C.)

| | | 28 days |
|---|---|---|
| Organism | Initial (CFU'S/mL) | (CFU's/mL) |
| Aspergillus niger | 10⁴ | 10⁴ |
| Bacillus cereus | 10⁶ | 10⁵ |
| Burkholderia cepacia | 10⁸ | 10⁶ |
| Candida albicans | 10⁶ | 10⁵ |
| Haemophilus influenzae | 10⁶ | 10⁵ |
| Pseudomonas aeruginosa | 10⁷ | 10⁵ |
| Staphylococcus aureus | 10⁸ | 10⁷ |
| Staphylococcus epidermidis | 10⁷ | 10⁶ |
| Streptococcus bovis | 10⁷ | 10⁵ |
| Streptococcus pyogenes | 10⁷ | 10⁵ |
| Streptococcus pneumoniae | 10⁶ | 10⁴ |

Interpretation:
Test results reported are based on “dry” streak methods (no rehydration fluid utilized).
Conclusions:
Device allows product to withstand constant stress temperature for up to 28 days and maintain viability with only a 1-2 log reduction.

TABLE 2
ACCELERATED STUDIES (STORAGE
TEMPERATURE: 30 C.)

| | | 1 | 2 | 4 | 6 |
|---|---|---|---|---|---|
| Organism | Initial | Month | Months | Months | Months |
| Streptococcus pyogenes | 4+ | 4+ | 4+ | 4+ | 3+ |
| Streptococcus aglactiae | 4+ | 4+ | 4+ | 4+ | 3+ |
| Escherichia coli | 4+ | 4+ | 4+ | 4+ | 3+ |
| Bacillus subtilis | 4+ | 4+ | 4+ | 4+ | 3+ |
| Staphylococcus aureus | 4+ | 4+ | 4+ | 4+ | 4+ |
| Haemophilus influenzae | 4+ | 4+ | 4+ | 4+ | 2+ |
| Streptococcus pneumoniae | 4+ | 4+ | 4+ | 4+ | 2+ |
| Enterococcus faecalis | 4+ | 4+ | 4+ | 4+ | 3+ |
| Klebsiella pneumonie | 4+ | 4+ | 4+ | 4+ | 3+ |
| Rhodococcus equi | 4+ | 4+ | 4+ | 4+ | 2+ |

Interpretation:
Viability Scale: 0 (No Growth), 1+ Growth in 1^{st} Quadrant, 2+ Growth in 2^{nd} Quadrant, 3+ Growth in 3^{rd} Quadrant, 4+ Growth in 4^{th} Quadrant.
Conclusions:
Device allows product to withstand constant room temperature conditions for up to 6 months and maintain easy recovery without pre-rehydration.

TABLE 3
(STORAGE TEMPERATURE: 2-8 C.)

| | | 1 | 5 | 9 | 15 |
|---|---|---|---|---|---|
| Organism | Initial | Month | Months | Months | Months |
| Streptococcus pyogenes | 4+ | 4+ | 4+ | 4+ | 3+ |
| Streptococcus aglactiae | 4+ | 4+ | 4+ | 3+ | 3+ |
| Escherichia coli | 4+ | 4+ | 4+ | 3+ | 3+ |
| Bacillus subtilis | 4+ | 4+ | 4+ | 4+ | 3+ |
| Staphylococcus aureus | 4+ | 4+ | 4+ | 3+ | 4+ |
| Haemophilus influenzae | 4+ | 4+ | 4+ | 3+ | 3+ |
| Streptococcus pneumoniae | 4+ | 4+ | 3+ | 3+ | 3+ |
| Enterococcus faecalis | 4+ | 4+ | 4+ | 3+ | 3+ |
| Klebsiella pneumonie | 4+ | 4+ | 4+ | 3+ | 3+ |
| Rhodococcus equi | 4+ | 4+ | 3+ | 2+ | 2+ |

Interpretation:
Viability Scale: 0 (No Growth), 1+ Growth in 1^{st} Quadrant, 2+ Growth in 2^{nd} Quadrant, 3+ Growth in 3^{rd} Quadrant, 4+ Growth in 4^{th} Quadrant.
Conclusions:
Device allows product to withstand constant refrigerated temperature conditions for up to 15 months and maintain easy recovery without pre-rehydration.

TABLE 4
COMPARISON STUDY STORAGE TEMPERATURE (2-8 C.)

| Organism | Format | 12 Months |
|---|---|---|
| Escherichia coli | Pellet | 2+ |
| 25922 | Fibrous Network | 3+ |
| Streptococcus pneumoniae | Pellet | 2+ |
| 49150 | Fibrous Network | 3+ |
| Staphylococcus aureus | Pellet | 2+ |
| 25923 | Fibrous Network | 3+ |
| Bacillus cereus | Pellet | 2+ |
| 11778 | Fibrous Network | 3+ |
| Campylobacter jejuni | Pellet | 0 |
| 33291 | Fibrous Network | 2+ |

Interpretation:
Viability Scale: 0 (No Growth), 1+ Growth in 1^{st} Quadrant, 2+ Growth in 2^{nd} Quadrant, 3+ Growth in 3^{rd} Quadrant, 4+ Growth in 4^{th} Quadrant.
Conclusions:
Fibrous network provides for improved recovery of viable cells versus a lyophilized pellet. Particularly with Campylobacter jejuni.

We claim:

1. A device for storing freeze-dried or otherwise desiccated microbials cells or cellular components intended for recovery of the microbial cells or cellular components for delivery of viable microbial cells, comprising;
a. a disposable inoculation device having a closely knitted fibrous network of Dacron, polyester, rayon, nylon, cotton or other natural or synthetic fibers for greater removal of bound water or free water during lyophilization or desiccation processes,
b. a quantity of reconstitutable freeze-dried or desiccated microbial cells or cellular components impregnated throughout said closely knitted fibrous network,
whereby said device can provide increased stability of freeze-dried or desiccated microbial cells or cellular components and a means of direct inoculation of freeze-dried or desiccated microbial cells and cellular components to solid or liquid growth culture or tissue culture media eliminating pre-rehydration steps.

2. The device of claim 1 further including a packaging for holding the device, comprising;
a. a desiccant,
b. a sterile atmosphere substantially free from water and oxygen;
c. a combination of plastic containers and metallic foils to contain said device.

3. The device of claim 1 further intended for recovery of eukaryotic cells.
